(19) United States
(12) Patent Application Publication
(10) Pub. No.: US 2021/0085893 A1
(43) Pub. Date: Mar. 25, 2021

(54) DEVICE AND METHODOLOGY FOR PREPARING SKIN FOR SELF-INJECTION

(71) Applicant: Danielle T. Abramson, Towaco, NJ (US)

(72) Inventor: Danielle T. Abramson, Towaco, NJ (US)

(21) Appl. No.: 17/032,059

(22) Filed: Sep. 25, 2020

Related U.S. Application Data
(60) Provisional application No. 62/905,514, filed on Sep. 25, 2019.

(51) Int. Cl. A61M 5/42

(52) U.S. Cl. CPC A61M 5/425; A61M 2209/082

(57) ABSTRACT

A device for pinching a user's skin includes a first head member including a handle portion, a jaw portion, and an intermediate portion therebetween, the handle portion having a terminal end with an engagement means; a second head member including a handle portion, a jaw portion, and an intermediate portion therebetween, the handle portion having an elongated end with a generally arcuate translating rack with a corresponding engagement means; and pivot means on the intermediate portions permitting the jaw portions to converge on the skinfold and grasp the skinfold in response to initial movement of the handle portions toward each other.

### RELATED APPLICATION

This application claims the benefit of and priority to U.S. provisional Application Ser. No. 62/905,514 filed Sep. 25, 2019, the entire disclosure of which is incorporated by reference in its entirety.

### BACKGROUND

Many patient medicines are in liquid form and need to be injected into the subcutis, the fatty layer of tissue just under the skin (subcutaneous injections, “SQ”). As subcutaneous tissue has few blood vessels, the injected drug is diffused very slowly at a sustained rate of absorption. Therefore, it is highly effective in administering vaccines, growth hormones, and insulin, which require continuous delivery at a low dose rate. For some pain and allergic medications, the subcutaneous tissue injections are the preferred choice. Typically, pinching a skinfold prior to injecting ensures that the drug is administered into the subcutaneous layer, as opposed to muscle.

Common subcutaneous sites are outer aspects of arms, thighs, buttocks and abdomen. Typically, individuals alternate the injection site each time to avoid soreness at any one sight. While it is fairly easy for an individual to pinch their abdomen and thighs with one hand and administer the injection with the other hand, it is quite difficult, and nearly impossible, for an individual to pinch their arm with one hand and administer the injection.

### SUMMARY

In one aspect, the present invention provides devices and methodology for preparing a skin for self-injection. The devices can be used to help an individual pinch himself when injecting medicine within a syringe. A device of the present invention is essentially a portable self-pincher device that can be used by an individual to pinch their skin so that the user then has access to both hands for administering a subcutaneous injection of medicine.

In one embodiment, a pinching device of the present disclosure includes a first head member including a handle portion, a jaw portion, and an intermediate portion therebetween, the handle portion having a terminal end with an engagement means; a second head member including a handle portion, a jaw portion, and an intermediate portion therebetween, the handle portion having an elongated end with a generally arcuate translating rack with an engagement means; and pivot means on the intermediate portions permitting the jaw portions to converge on the skin and grasp the skin in response to initial movement of the handle portions toward each other, thus creating a skinfold.

Further, a method for preparing a fold of skin of a user for self-injection is disclosed that comprises placing a pinching device of the present disclosure in an open position over the user's fold of skin, wherein in the open position, the terminal end of the first head member is disengaged with the rack of the second head member, thus creating a space between the jaw portions that is large enough to engulf the area of skin that requires pinching; squeezing, using a hand of the user, the handle portion of the first head member towards the handle portion of the second head member, wherein the engagement means on the terminal end of the handle portion of the first head member lock into position with the corresponding engagement means on the translating rack of the handle portion of the second head member; and creating a skinfold.

In one embodiment, a pinching device of the present disclosure includes two opposing clamping surfaces formed on upper ends of elongated rigid arms that are joined on lower ends by a resilient member such that the clamping surfaces are movable relative to one another through a fixed radius about a common pivot point, wherein the rigid arms and clamping surfaces are biased away from each other by the resilient member. In an embodiment, at least one of the rigid arms of the pinching device includes a locking mechanism sufficiently designed to lock the rigid arms in a compressed state so that the clamping surfaces are squeezed together and can squeeze a skin and lock in position so that a user will then have free use of both hands after creating a skinfold.

Also disclosed herein is a kit for preparing skin for a self-injection that includes a pinching device disclosed herein; a pair of pads for placement on the jaw portions or clamping portions of the pinching device; a flexible gooseneck arm; and a clip. In an embodiment, the kit is housed in a container.

### BRIEF DESCRIPTION OF THE DRAWINGS

FIG. 1 shows an embodiment of a device of the present invention during use. In this embodiment, the device includes a moveable arm having a first end with a clip integrated therein and a second end with a pinching device. Although the pinching device of the first embodiment is shown in FIG. 1, any of the pinching devices disclosed herein can be used with the clip and moveable arm.

FIG. 2 is a perspective view of the device of FIG. 1.

FIG. 3 is a side elevational view of a first embodiment of a pinching device of the present invention in an open state.

FIG. 4 is a side elevational view of the first embodiment of the pinching device of FIG. 3 in a pinched state.

FIG. 5 is a perspective view of a second embodiment of a pinching device of the present invention in an open state.

FIG. 6 is a perspective view of the second embodiment of the pinching device of FIG. 5 in a pinched state.

FIG. 7 is a perspective view of a third embodiment of a pinching device of the present invention.

FIG. 8 is a perspective view of the third embodiment of the pinching device of FIG. 7 in a pinched state.

FIG. 9 is a partial, enlarged view of the jaw portions of the pinching device of FIG. 8.

FIG. 10 shows an embodiment of a device of the present invention. In this embodiment, the device includes a moveable arm having a first end with a clip integrated therein and a second end with the pinching device of FIG. 7.

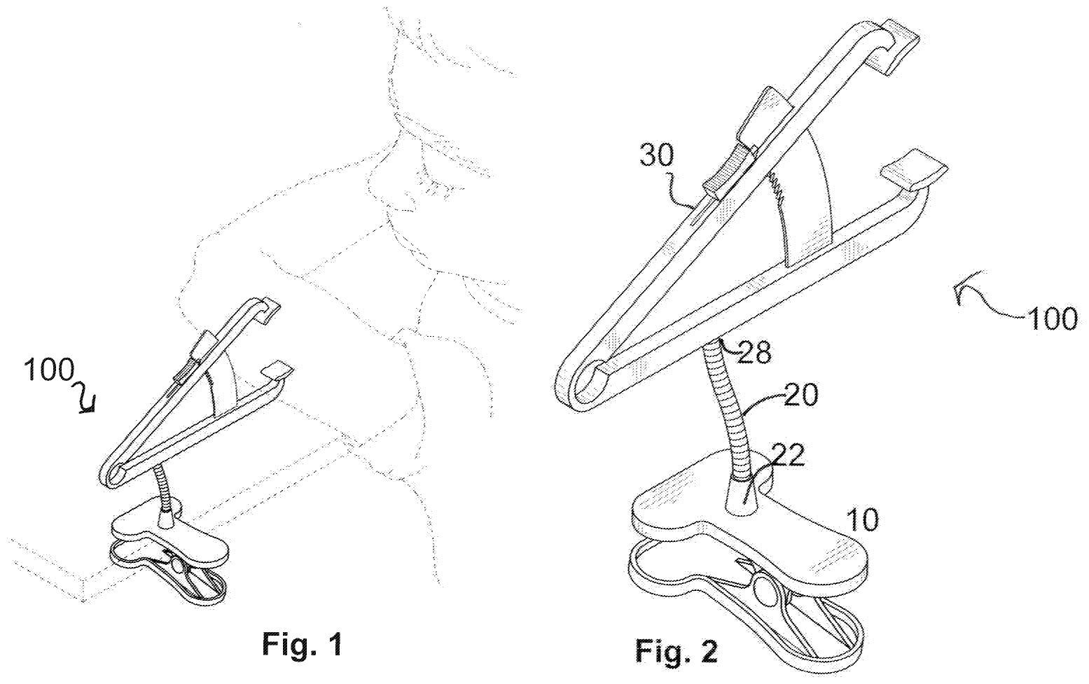

The human body and desk surface shown in broken lines in FIG. 1 is to show environmental and form no part of the claimed design.

### DETAILED DESCRIPTION

FIG. 1 in conjunction with FIG. 2 shows an embodiment of a device 100 of the present invention. Device 100 includes a moveable arm 20 having a first end 22 with a clip 10 integrated therein and a second end 28 with a pinching device 30 with rotational adjustment. The clip 10 is sufficiently designed to couple to a surface, such as a table or desk, which is illustrated in FIG. 1 in broken lines. Although the clip 10 is secured to a table in FIG. 1, the clip 10 can be secured to any surface that gives the device 100 support. The clip 10 can be any device that can secure the device 100 to a surface. Although pictured as a clip 10, in another example, the device 100 can be secured to a surface by magnets, screws, Velcro, a pressure sensor, a machine, hydraulics, or any other securing means. The pinching device 30 is essentially a portable self-pincher device that can be used by an individual to pinch their skin so that the user then has access to both hands for administering a subcutaneous injection of medicine. In an embodiment, the moveable arm 20 is a flexible gooseneck arm. In an embodiment, the moveable arm 20 is an articulating arm. Preferably, the flexible moveable arm 20 is constructed of a material such that once bent, the moveable arm 20 stays in that position. The pinching device 30 can be used alone or together with the clip/moveable arm component.

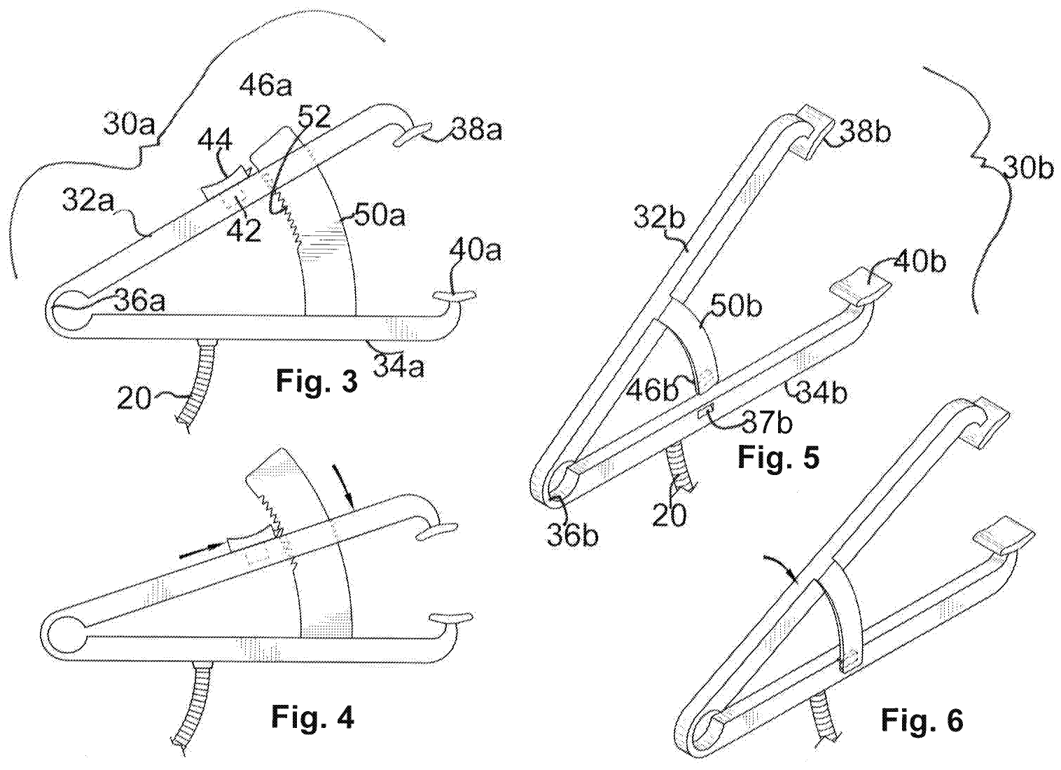

FIG. 3 and FIG. 4 show a first embodiment of a pinching device 30a. Pinching device 30a includes two opposing clamping surfaces 38a, 40a formed on the upper ends of elongated rigid arms 32a, 34a. Arms 32a, 34a are joined on the lower ends by resilient member 36a. Clamping surfaces 38a, 40a are thus movable relative to one another through a fixed radius about a common pivot point. Rigid arms 32a, 34a and clamping surfaces 38a, 40a are biased away from each other by resilient member 36a. The skin of a person is manually compressed or pinched by clamping surfaces 38a, 40a by the applying pressure on arms 32a, 34a, thus creating a skinfold.

Rigid arm 32a includes thumb locking mechanism 44 that is moveable within a slot 42 on the rigid arm 32a. Thumb locking mechanism 44 can be positioned anywhere on the rigid arm 32a. Thumb locking mechanism 44 can be a button or any device that the individual can use to lock the arms 32a and 34a in place. The movable locking mechanism 44 can be locked when the individual has a position that feels comfortable and the individual wants to secure the pinching device 100.

Arc arm 50a extends from arm 32a inward towards arm 34a. Arc arm 50a has an arcuate shape at approximately the same arc as the arc of the pivoting motion of clamping surfaces 38a, 40a. The arc arm 50a includes a plurality of saw tooth indents 52. A projection 46a extends from thumb locking mechanism 44 and can engage with a saw tooth indent 52 on the arc arm 50a as the clamping surfaces 38a, 40a are moved toward one another by compression of rigid arm 32a and 40a, as illustrated in FIG. 4. In an embodiment, the engagement of projection 46a into saw tooth indent 52 emits an audible “click”. The audible “click” will serve as an indication of the appropriate pressure level. The projection 46a removably locks into position within saw tooth indent 52.

FIG. 5 and FIG. 6 show a second embodiment of a pinching device 30b. Pinching device 30b includes two opposing clamping surfaces 38b, 40b formed on the upper ends of elongated rigid arms 32b, 34b. Arms 32b, 34b are joined on the lower ends by resilient member 36b. Clamping surfaces 38b, 40b are thus movable relative to one another through a fixed radius about a common pivot point. Rigid arms 32b, 34b and clamping surfaces 38b, 40b are biased away from each other by resilient member 36b. The skin of a person is manually compressed or pinched by clamping surfaces 38b, 40b by the applying pressure on arms 32b, 34b, thus creating a skinfold.

Arc arm 50b extends from arm 32b inward towards arm 34b. Arc arm 50b has an arcuate shape at approximately the same arc as the arc of the pivoting motion of clamping surfaces 38b, 40b. Rigid arm 34b includes a hole 37b which is sufficiently designed to engage with a ledge 46b on arc arm 50b. Ledge 46b can engage with hole 37b on rigid arm 34b as the clamping surfaces 38b, 40b are moved toward one another by compression of rigid arm 32b and 40b, as illustrated in FIG. 6. The ledge 46b removably locks into position within hole 37b.

Although shown in FIG. 5 and FIG. 6 as having one hole 37b, in one embodiment, the rigid arm 34b can have the hole 37b in any location on the rigid arm 34b. In another embodiment, the rigid arm 34b can have multiple holes 37b to change the position of the clamping surfaces 38b, 40b. In this embodiment, the clamping surfaces 38b, 40b would have to move or slide on the arm 32b or the rigid arm 34b (or both).

In an embodiment of use, when the pinching device 30 is used in conjunction with the clip/moveable arm, an individual would connect clip 10 of device 100 to a surface, the individual will adjust the arm 20 so that pinching device 30 is positioned in close proximity to a desired portion of the skin. Using the pinching device 30, the desired portion of the skin is grasped between the clamping surfaces 38, 40 by applying a compressive pressure over skin by squeezing rigid arms 32 and 40 together, thus creating a skinfold. Once clamping surfaces 38, 40 reach a pressure level, the pinching device 30 can be removably locked into position. In an embodiment, this engagement results in an interim lock which allows the user to fully remove their hands from the pinching device and skinfold so that the user has free use of both hands. The user may now use their free hand to inject themselves with medicine. Once the medicine has been administered, the user releases the locking mechanism releasing arms 32, 34 and clamping surfaces 38 and 40 are removed from skinfold 60. It should be noted that a pinching device of the present invention could be used alone, without the aid of the clip/moveable arm.

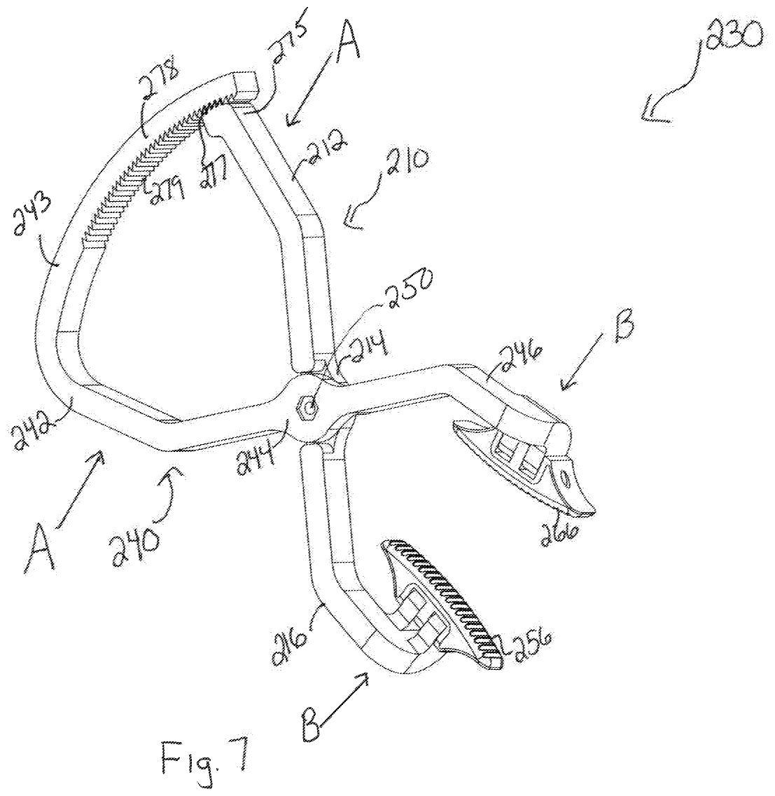

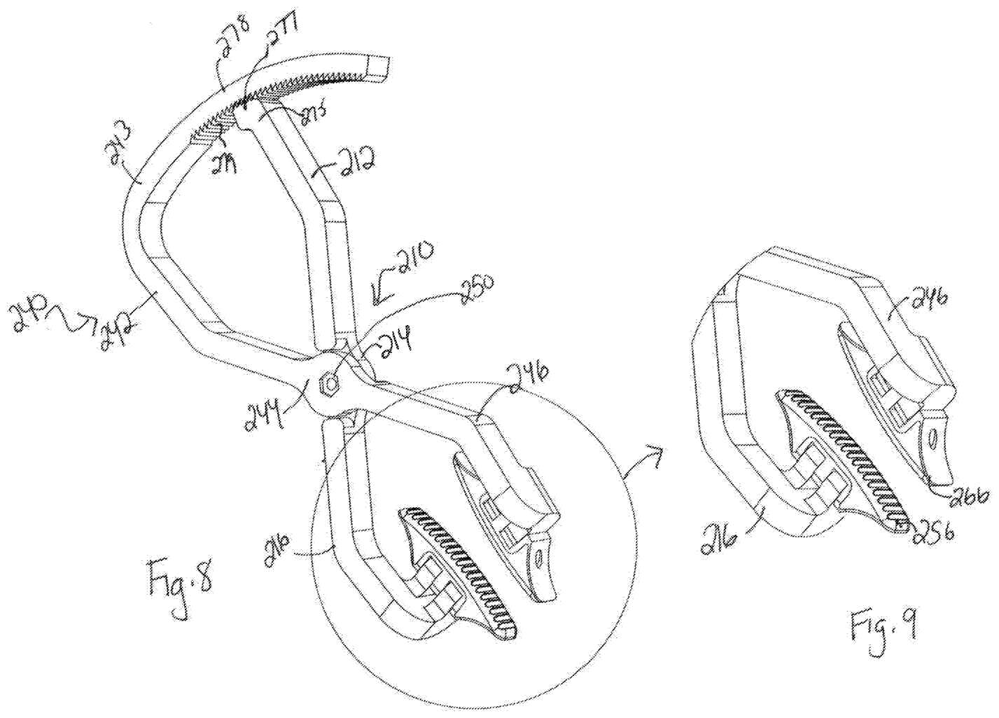

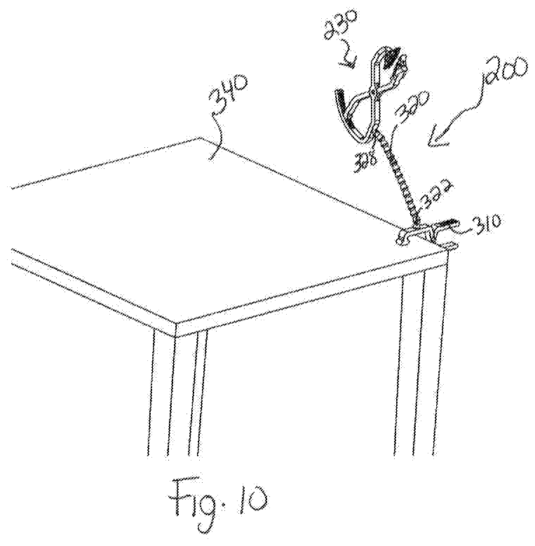

FIG. 7 and FIG. 8 show a third embodiment of a pinching device 230 of the present invention. Pinching device 230 includes a first head member 210 and a second head member 240 interconnected in a scissor-like arrangement in order to easily and efficiently adjust in size. The first head member 210 includes a handle portion 212, an intermediate portion 214, and a jaw portion 216. Similarly, the second head member 240 includes a handle portion 242, an intermediate portion 244, and a jaw portion 246. Jaw portions 216 and 246 include pads 256 and 266, which may be removably attached to each jaw portion, respectively. The pads 256 and 266 provide a soft surface for pinching a skinfold and can be made from a medical grade plastic or similar material. A close-up of the jaw portions 216 and 246 with the pads 256 and 266 are illustrated in FIG. 9. In an embodiment, the pads 256 and 266 are removably attached to the jaw portions. In an embodiment, the pads 256 and 266 are rotationally attached to the jaw portions,

Handle portions 212 and 242 are movable about a central pivot 250 which passes centrally through intermediate portion 214 and intermediate portion 244. Handle 242 includes a generally arcuate translating rack 278 formed on an elongated end 243 of the handle 242. Handle 212 has a terminal end 275. The rack 278 includes engagement means, illustrated as a plurality of teeth 279 and the terminal end 275 also includes complimentary engagement means, illustrated as a plurality of teeth 277. Means to secure intermediate portions 214 and 244 generally comprises a screw aligned with slot 250 formed on each of the intermediate portions 214 and 244.

In an open position, the terminal end 275 of the handle 212 would not engage with the main rack 278 and the jaw portions 216 and 246 will be spread apart a distance that allows a portion of the skin that requires pinching to be within the opening created by the jaw portions 216 and 246. FIGS. 7 and 8 show what happens when force is applied to the handle portions 212 and 242. Applying a force (compressive pressure) to the handle portions 212 and 242, causes the handle portions 212 and 242 to move toward each other as indicated by arrows A and jaw portions 216 and 246 to move toward each other as indicated by arrows B to compress or pinch the skinfold of a person. This in turn results in the terminal end 275 moving along the main rack 278. Once the pads 256 and 266 of j aw portions 216 and 246 engage the skin, the teeth 277 on the terminal end 275 are forced into engagement with the plurality of teeth 279 formed on the rack 278. The pinching device 230 is now in a locked position. This engagement results in an interim lock which allows the user to fully remove their hands from the pinching device 230 and resulting skinfold created so that the user has free use of both hands. The user may now use their free hand to inject themselves with medicine. Once the medicine has been administered, the user pulls apart the handle portions 212 and 242 which releases the engagement of the teeth 277 and 279 and jaw portions 216 and 246 are removed from the skinfold.

FIG. 10 shows an embodiment of a device 200 of the present invention. Device 200 includes a moveable arm 320 having a first end 322 with a clip 310 integrated therein and a second end 328 with the pinching device 230. It should be noted that the pinching device 230 could be used alone, without the aid of the clip/moveable arm. The clip 310 is sufficiently designed to couple to a surface, such as a table or desk 340. Although the clip 310 is secured to a table 340, the clip 310 can be secured to any surface that gives the device 200 support. The clip 310 can be any device that can secure the device 200 to a surface. Although pictured as a clip 310, in another example, the device 200 can be secured to a surface by magnets, screws, Velcro, a pressure sensor, a machine, hydraulics, or any other securing means. In an embodiment, the moveable arm 320 is a flexible gooseneck arm. In an embodiment, the moveable arm 320 is an articulating arm. Preferably, the flexible moveable arm 320 is constructed of a material such that once bent, the moveable arm 320 stays in that position. The pinching device 230 can be used alone or together with the clip/moveable arm component.

### Kits

In embodiments, a pinching device of the present invention is part of a kit that further includes a pair of pads for placement on the jaw portions or clamping surfaces of the pinching device; a flexible gooseneck arm; a clip; and a container for housing the items. In some embodiments, the kit further includes one or more instruction sets for instructing the user on how to use the device, specialized packaging, labels, decorative designs, coupons, and the like.

Those skilled in the art should appreciate that other improvements are possible and envisioned and fall within the scope of the invention.

It is accordingly intended that all matter contained in the above description or shown in the accompanying drawings be interpreted as illustrative rather than in a limiting sense.

What is claimed is:

1. A device for pinching a user's skin comprising:
a first head member including a handle portion, a jaw portion, and an intermediate portion therebetween, the handle portion having a terminal end with an engagement means;
a second head member including a handle portion, a jaw portion, and an intermediate portion therebetween, the handle portion having an elongated end with a generally arcuate translating rack with a corresponding engagement means; and
pivot means on the intermediate portions permitting the jaw portions to converge on the skinfold and grasp the skinfold in response to initial movement of the handle portions toward each other.

2. The device of claim 1, wherein each of the jaw portions include pads.

3. The device of claim 2, wherein the pads are removably attached to the jaw portions.

4. The device of claim 2, wherein the pads are rotationally attached to the jaw portions.

5. The device of claim 1, wherein the engagement means at the terminal end the first head member is a plurality of teeth and the engagement means at the rack on the second head member are corresponding teeth.

6. The device of claim 1 operatively connected to a flexible gooseneck arm.

7. The device of claim 6, wherein the flexible gooseneck arm is operatively connected to a clip.

8. A kit for preparing skin for self-injection comprising:
(a) the device of claim 1;
(b) a pair of pads for placement on the jaw portions;
(c) a flexible gooseneck arm;
(d) a clip; and
(e) a container for housing items (a)-(d).

9. The kit of claim 8, wherein the container is a box.

10. The kit of claim 8, wherein the container is a bag.

11. The kit of claim 8, further comprising an instruction set.

12. A method for preparing a fold of skin of a user for self-injection comprising:
placing the device of claim 1 in an open position over the user's fold of skin, wherein in the open position, the terminal end of the first head member is disengaged with the rack of the second head member, thus creating a space between the jaw portions that is large enough to engulf the area of skin that requires pinching;
squeezing, using a hand of the user, the handle portion of the first head member towards the handle portion of the second head member, wherein the engagement means on the terminal end of the handle portion of the first head member lock into position with the corresponding engagement means on the translating rack of the handle portion of the second head member; and
creating a skinfold.

13. The method of claim 12, further comprising removing the hand of the user from the device so that the device is left independently squeezing the skinfold of the user.

14. The method of claim 14, further comprising, injecting medicine into the skinfold using the same hand that was previously squeezing the device.
